(19) United States
(12) Patent Application Publication
(10) Pub. No.: US 2015/0164703 A1
(43) Pub. Date: Jun. 18, 2015

(54) EXCRETA DETECTING SENSOR AND DETECTING DEVICE USING ELECTRICALLY-CONDUCTIVE FIBROUS CONDUCTING WIRE

(71) Applicant: ITHEALTH CO., LTD., Daegu (KR)

(72) Inventor: Yun Seob Bae, Daegu (KR)

(21) Appl. No.: 14/406,488

(22) PCT Filed: Jun. 17, 2013
(86) PCT No.: PCT/KR2013/005310

§ 371 Date: Dec. 8, 2014

(30) Foreign Application Priority Data
Jun. 15, 2012 (KR) 10-2012-0064380
Jun. 17, 2013 (KR) 10-2013-0068908

(51) Int. Cl. A61F 13/42; G01N 27/04

(52) U.S. Cl. CPC A61F 13/42; G01N 27/048; A61F 2013/426; A61F 2013/424

(57) ABSTRACT

The present invention relates to an excreta detecting sensor for detecting excrement/urine from people who are unable to control their own urination, such as elderly individuals, disabled individuals, patients and infants, and relates to an excreta detecting apparatus which automatically notifies a carer of the urination. The excreta detecting sensor according to the present invention includes a conductive fiber having elasticity and absorbent polymer having the characteristic of expanding upon absorbing moisture such as moisture absorbent used in diaper. The excreta detecting apparatus according to the present invention wirelessly notifies the user of the excreta detection information resulting from the defecation or the urination from diaper in which the excreta detecting sensor is mounted.

### TECHNICAL FIELD

The present invention relates to a technology of detecting excrement resulting from defecation or urine resulting from urination for the convenience of people who are unable to dispose urine and excrement for themselves and notifying the defecation or the urination to the people of the carers. More specifically, the present invention relates to an excreta detecting sensor employing a fiber conductive line, capable of detecting detect excrement or urine using conductive fiber having elasticity and super absorbent polymer representing superior moisture absorbency, and an excreta detecting apparatus employing a fiber conductive line, capable of detecting the excrement or the urine by the excreta detecting sensor to notify defecation or urination to a carer.

### BACKGROUND ART

In general, people who cannot dispose excrement resulting from defecation or urine resulting from urination for themselves may wear diaper, and a carer may collect and dispose the diaper used in the defecation or the urination. In this case, if the carer personally sees the diaper with the naked eyes of the carer to determine whether a user defecates or urinates, the carer may be significantly bothered with the seeing of the diaper.

Further, if the carer does not check the defecation or urination at the right time, since the user (cared person) has worn the diaper having the excrement resulting from the defecation or urine resulting from the urination for a long time, the cared person becomes in an unsanitary state and the unsanitary state may exert a harmful influence on the health of the cared person.

Therefore, recently, research and development have been actively performed on an excreta detecting sensor to detect the defecation or the urination of the cared person and an apparatus for detecting the defecation or the urination by the cared person using the excreta detecting sensor.

However, conventionally, a resistive type excreta detecting sensor has been mainly used to detect the defecation or the urination based on moisture contained in the excrement or the urine. The resistive type excreta detecting sensor has a structure of exposing the contact surface with the excrement or the urine to detect the moisture contained in the excrement or the urine.

Therefore, when the conventional resistive-type excreta detecting sensor is used, and when the cared person has worn the diaper for a long time after the defecation or the urination, foreign matters may stick to the contact surface of the excreta detecting sensor with the excrement or the urine or the contact surface may be oxidized, so that the ability of the excreta detecting sensor to detect the excreta may be degraded.

In addition, when the conventional resistive-type excreta detecting sensor is used, and when the cared person moves with the diaper after the defection or the urination, an amount of moisture existing on the surface of the sensor is varied so that the reliability may be degraded in detecting the excreta.

Further, when the conventional resistive-type excreta detecting sensor is used, and when the moisture contained in the excrement or the urine makes contact with a metallic surface, a polarizing phenomenon occurs, so that a resistance component is increased. Therefore, conductivity may be degraded.

In addition, when the conventional resistive-type excreta detecting sensor is used, the contact surface of the excreta detecting sensor with the excrement or the urine is directly exposed to the excrement or the urine so that the cared person becomes in an unsanitary state. In addition, the conventional resistive-type excreta detecting sensor is sensitive to the change of surrounding environments and high at a price.

Although various sensors such as a gas sensor or a temperature sensor are applied the conventional diaper, the sensors are significantly high at price, so that the sensors may not be extensively spread.

### DISCLOSURE

### Technical Problem

An object of the present invention is to provide an excreta detecting sensor capable of easily being mounted into diaper in a simpler structure substantially without the harm to a user of the diaper.

Another object of the present invention is to provide an excreta detecting apparatus capable of transmitting an excreta signal, which is detected by the excreta detecting sensor, to a carer through wireless and wired transceiving paths.

### Technical Solution

In order to accomplish the above objects, according to one aspect of the present invention, there is provided an excreta detecting sensor employing a fiber conductive line, which includes a plurality of fiber conductive lines arranged in parallel to each other while being spaced apart from each other by a predetermined interval, a plurality of superior absorbent polymer sheets spaced apart from each other by a predetermined distance, and arranged on or under the fiber conductive lines perpendicularly to the fiber conductive lines while making contact with the fiber conductive lines, so that each fiber conductive line is changed to a conductive state or maintained in an insulating state depending on defecation or urination of a diaper wearer, a power terminal connected with one of the fiber conductive lines to output excreta detection signal resulting from the defecation or the urination, and an output terminal connected with at least one of remaining fiber conductive lines.

According to another aspect of the present invention, there is provided an excreta detecting sensor employing a fiber conductive line, which includes a first fiber conductive line part including superior absorbent polymer which connects first and second fiber conductive lines having predetermined elasticity or more with each other and is gelated by moisture resulting from defecation or urination of a diaper wearer to disconnect the first and second fiber conductive lines from each other, a second fiber conductive line part having a same structure as a structure of the first fiber conductive line part, a fiber conductive line connecting part to connect the second fiber conductive line of the first fiber conductive line part with a second fiber conductive line of the second fiber conductive line part, and a sensor connecting part comprising a power supply unit connected with the first fiber conductive line of the first fiber conductive line part and an excreta detection signal output terminal connected with the first fiber conductive line of the second fiber conductive line part.

According to still another aspect of the present invention, there is provided an excreta detecting apparatus employing a fiber conductive line, which includes an excreta detecting sensor that detects moisture resulting from defecation or urination of a diaper wearer to output an excreta detection signal according to moisture detection, a transmitter that creates excreta detection information from the excreta detection signal output from the excreta detecting sensor to wirelessly transmit the excreta detection information, and a receiver that receives the excreta detection information from the transmitter and visibly or acoustically represents the defecation or urination of the diaper wearer.

### Advantageous Effects

As described above, the present invention can provide the economical excreta detecting sensor having the simpler structure substantially without the harm to the user and the excreta detecting apparatus including the excreta detecting sensor, thereby exactly notifying the defecation or the urination to a carer who is located at a long distance as well as a short distance within a short period of time.

In addition, different from a conventional excreta detecting sensor, the excreta detecting sensor according to the present invention includes conductive fiber having elasticity and absorbent polymer having a characteristic of expanding the volume thereof and a gelation characteristic when absorbing water, thereby exactly detecting various types of excreta.

### DESCRIPTION OF DRAWINGS

FIG. 1 is a schematic view showing an excreta detecting sensor according to a first embodiment of the present invention.

FIG. 2a is a schematic view showing an excreta detecting sensor according to a second embodiment of the present invention.

FIG. 2b (a)-(c) illustrates structures of a first fiber conductive line for the explanation thereof.

FIG. 2c is a view showing an excreta detecting sensor produced in a band form in the manufacturing process thereof for the explanation thereof.

FIG. 3 is a schematic view showing an example that the excreta detecting sensor according to the present invention is mounted into diaper.

FIG. 4 is a schematic view showing an excreta detecting apparatus according to a third embodiment of the present invention.

FIG. 5 is a block diagram showing a transmitter according to one embodiment.

FIG. 6 is a block diagram showing a receiver according to one embodiment.

### BEST MODE

### Mode for Invention

Hereinafter, an exemplary embodiment of the present invention will be described in detail with reference to accompanying drawings.

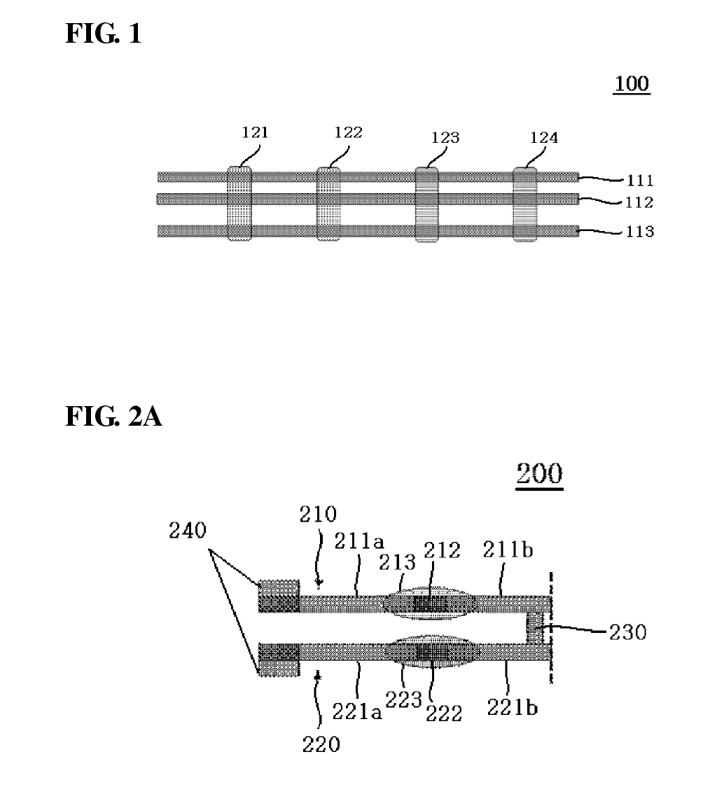

FIG. 1 is a schematic view showing an excreta detecting sensor according to a first embodiment of the present invention. As shown in FIG. 1, an excreta detecting sensor 100 includes first to third conductive lines 111 to 113 and first to fourth superior absorbent polymer sheets 121 to 124.

In this case, super absorbent polymer, which represents moisture absorbency superior to that of a conventional resin sheet, may be applied onto the surfaces of the first to fourth superior absorbent polymer sheets 121 to 124. Accordingly, even if the excreta detecting sensor 100 is stored for a long period of time after the excreta detecting sensor 100 has been shipped from a manufacturer, the contact surface of the excreta detecting sensor 100 with the super absorbent polymer is not prevented from being oxidized. In addition, since moisture absorbency is significantly highly represented, only an extremely small amount of moisture is evaporated from the surface of diaper even if a user (cared person) who wears diaper moves. The diaper includes disposable diaper.

In addition, before the super absorbent polymer is applied to the first to fourth superior absorbent polymer sheets 121 to 124, the contact surfaces of the first to fourth superior absorbent polymer sheets 121 to 124 with the first to third fiber conductive lines 111 to 113 may be coated with a material such as AgCl. Therefore, polarization is reduced on the contact surfaces of the first to fourth superior absorbent polymer sheets 121 to 124 with the first to third fiber conductive lines 111 to 113, so that moisture sensing ability is improved.

The excreta detecting sensor 100 may be fixedly mounted in diaper during a diaper production process and shipped, or attached to the diaper at a time point required by a user.

Referring to FIG. 1, the first to third fiber conductive lines 111 to 113 are arranged in parallel to each other while being spaced apart from each other by a predetermined distance. In this state, the first to fourth superior absorbent polymer sheets 121 to 124 are arranged on or under the first to third fiber conductive lines 111 to 113 in contact with the first to third fiber conductive lines 111 to 113. In this case, the first to fourth superior absorbent polymer sheets 121 to 124 are arranged perpendicularly to the first to third fiber conductive lines 111 to 113 while being spaced apart from each other by a predetermined distance.

In the state that a cared person who wears the diaper having the excreta detecting sensor 100 mounted into the diaper does not defecate or urinate, an amount of moisture contained in the first to fourth superior absorbent polymer sheets 121 to 124 of the excreta detecting sensor 100 is maintained at a predetermined value or less. Accordingly, the first to third fiber conductive lines 111 to 113 becomes insulated from each other.

However, if the cared person does defecation or urination, moisture resulting from the defecation or the urination is rapidly absorbed into at least one of the first to fourth superior absorbent polymer sheets 121 to 124, so that at least two of the first to third fiber conductive lines 111 to 113 are conducted to each other by the at least one of the first to fourth superior absorbent polymer sheets 121 to 124.

In addition, since power supply voltage is supplied to at least one of the first to third fiber conductive lines 111 to 113 from a power terminal connected with the at least one of the first to third fiber conductive lines 111 to 113 constantly (or at a predetermined period), the excreta detecting sensor 100 may output an excreta detection signal through an output terminal connected with at least one of the remaining first to third fiber conductive lines 111 to 113.

For example, in the state that the power supply voltage is supplied from the power terminal connected with the first fiber conductive line 111, if the cared person urinates, the moisture resulting from the urination may be rapidly absorbed into the second superior absorbent polymer sheet 122. In this case, the power supply voltage may be transmitted to the second fiber conductive line 112, the third fiber conductive line 113, or both of the second and third fiber conductive lines 112 and 113 through the first superior absorbent polymer sheet 122. The voltage, which is transmitted through the output terminal (not shown) connected with the second fiber conductive line 112 or the fiber conductive line 113, may be output as the excreta detection signal.

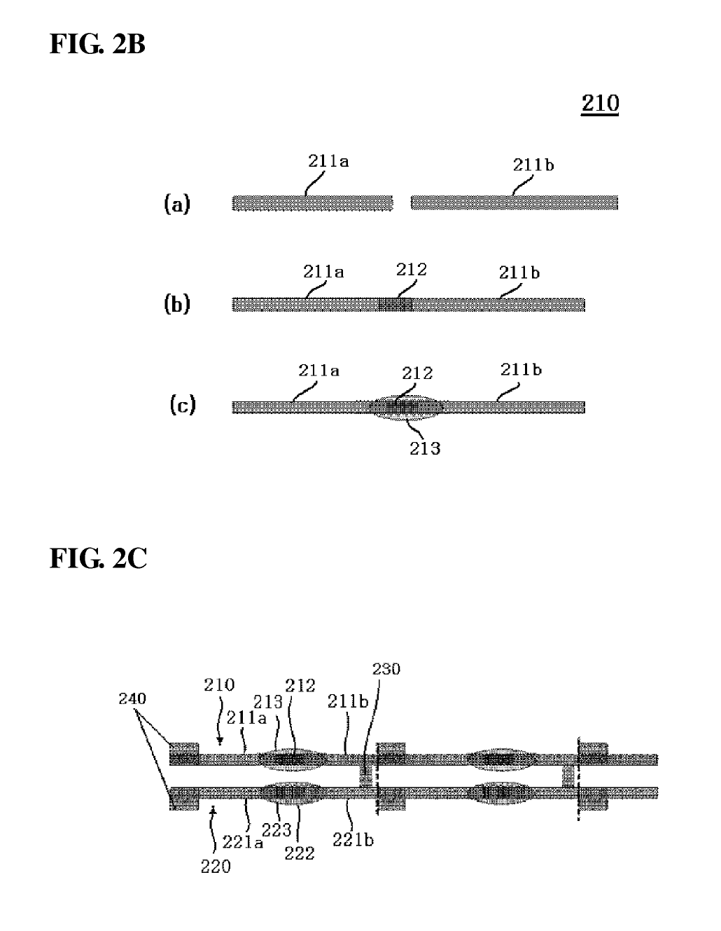

FIG. 2a is a schematic view showing an excreta detecting sensor according to a second embodiment of the present invention. As shown in FIG. 2a, an excreta detecting sensor 200 includes first and second fiber conductive line parts 210 and 220, a fiber conductive line connecting part 230, and sensor connecting parts 240.

The excreta detecting sensor 200 may be fixedly mounted in diaper during a diaper production process and shipped, or may be attached to the diaper at a time point required by a user.

The first fiber conductive line part 210 has the same structure as that of the second fiber conductive line part 220. FIG. 2b is a view to explain the structure of the first fiber conductive line part 210 for the illustrative purpose, and the first fiber conductive line part 210 includes first and second fiber conductive lines 211a and 211b and a fiber conductive line connecting part 213 including a superior absorbent polymer 212. The superior absorbent polymer may include resin used to absorb moisture in diaper, sanitary napkin, and the like.

The first and second fiber conductive lines 211a and 211b have more than predetermined elasticity. To this end, the first and second fiber conductive lines 211a and 211b may be fabricated through the same process as that applied to the fabrication of cloth having a spandex characteristic. For the conductivity of the first and second fiber conductive lines, the first and second fiber conductive lines may be coated with a conductive material such as silver (Ag) in the form of a thin film and fabricated, or may be fabricated in the form of a thread using a material having conductivity due to a complex compound.

The superior absorbent polymer 212 is provided as a connection material of the fiber conductive line connecting part 213. As shown in FIG. 2b (a)-(c), the first and second fiber conductive lines 211a and 221b are fixed to each other by the superior absorbent polymer 212 in the state that the first and second fiber conductive lines 211a and 211b closely makes contact with each other for the electrical conduction between the first and second fiber conductive lines 211a and 211b.

However, the superior absorbent polymer 212 excellently absorbs moisture, and is gelated after absorbing moisture to some extent. The first and second fiber conductive lines 211a and 211b are fixed by the superior absorbent polymer 212, which is solidified after the moisture is removed from the superior absorbent polymer 212, so that the connection state between the first and second fiber conductive lines 211a and 211b may be maintained. Through the above structure, the second fiber conductive line 211b of the first conductive line part 210 is connected with the second fiber conductive line 211b of the second fiber conductive line part 220 through the fiber conductive line connecting part 230. The sensor connecting parts 240 are connected with one side of the first fiber conductive line 211a of the first fiber conductive line part 210 and one side of the first fiber conductive line 221a of the second fiber conductive line part 220.

Power supply voltage having a predetermined level is supplied to the power terminal (not shown) provided at one side of the sensor connecting part 240. Therefore, the excreta detection signal resulting from the defecation or the urination of the cared part may be output through an output terminal provided at an opposite side of the sensor connecting part 240 as described below.

In the state that the cared person does not do defecation or urination, the first and second fiber conductive lines 211a and 211b of the first fiber conductive line part 210 are continuously connected with each other as described above by the superior absorbent polymer 212. Similarly, the first and second fiber conductive lines 221a and 221b of the second fiber conductive line part 220 are continuously connected with each other by superior absorbent polymer 222.

Therefore, the power terminal provided at one side of the sensor connecting part 240 is connected with the output terminal provided at an opposite side of the sensor connecting part 240 through the first fiber conductive line 211a of the first fiber conductive line part 210, the superior absorbent polymer 212, the second fiber conductive line 211b, the second fiber conductive line 221b of the second fiber conductive line part 220, the superior absorbent polymer 212, and the first fiber conductive line 221a, so that the excreta detection signal is output at a high level of more than a predetermined value. The high-level excreta detection signal represents that excrement or urine is not detected because the moisture resulting from the defecation or the urination is not detected.

However, in this state, if the cared person urinates, the moisture resulting from the urination is rapidly absorbed into the superior absorbent polymer 212 of the first fiber conductive line part 210. Therefore, the superior absorbent polymer 212 is gelated, so that the bonding strength in the connection part between the first and second fiber conductive lines 211a and 211b is degraded to or approximates the elasticity or less of the first and second fiber conductive lines 211a and 211b. Accordingly, the superior absorbent polymer 212, which connects the first and second fiber conductive lines 211a and 211b with each other, is broken. As well, the superior absorbent polymer 222, which connects the first and second fiber conductive lines 221a and 221b of the second fiber conductive line part 220 with each other, is broken.

Therefore, the excreta detection signal is output at a low level of a predetermined value or less through the path from the power terminal provided at one side of the sensor connecting part 240 to the output terminal provided at the opposite side of the sensor connecting part 240 in the state the cared person does the defecation or the urination. The low-level excreta detection signal represents that the excreta detection signal resulting from the defecation or the urination is output.

For reference, FIG. 2c shows an example that the excreta detecting sensor 200 having the above structure is produced in the form of a seamless band in the fabrication process.

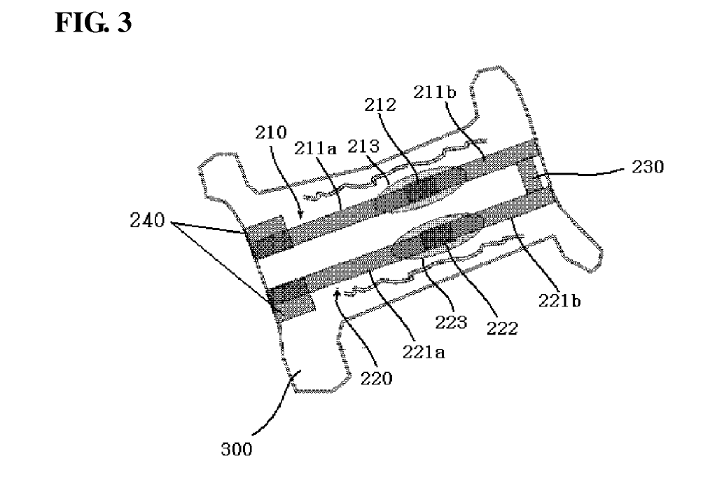

FIG. 3 is a schematic view showing an example that the excreta detecting sensor according to the present invention is mounted into the diaper. As shown in FIG. 3, diaper 300 and the excreta detecting sensor 200 are provided.

Referring to FIG. 3, the excreta detecting sensor 200 is attached to the diaper 300 along the longitudinal central line of an outer skin of the diaper 300, so that the superior absorbent polymer 212 of the first fiber conductive line part 210 are positioned together with the superior absorbent polymer 222 of the second fiber conductive line part 220 at the center of the diaper 300. Accordingly, the excrement from the defecation of the cared person and the urine from the urination of the cared person are detected within a short period of time as described above, so that the excreta detection signal resulting from the defecation or the urination can be output.

Preferably, when the excreta detecting sensor 200 is attached to the outer skin of the diaper 300, the first and second fiber conductive line parts 210 and 220 are mechanically elongated to some extent for the attachment to the diaper 300. Therefore, when the cared person does the defecation or the urination, the superior absorbent polymer 212 of the first fiber conductive line part 210 is gelated, so that the first and second fiber conductive lines 211a and 211b are easily disconnected from each other due to elasticity. Similarly, the superior absorbent polymer 222 of the second fiber conductive line part 220 is gelated, so that the first and second fiber conductive lines 221a and 221b are easily disconnected from each other due to the elasticity.

Although FIG. 3 shows the excreta detecting sensor according to the second embodiment for the illustrative purpose, the present invention is not limited thereto. The excreta detecting sensor according to the first embodiment may be provided instead.

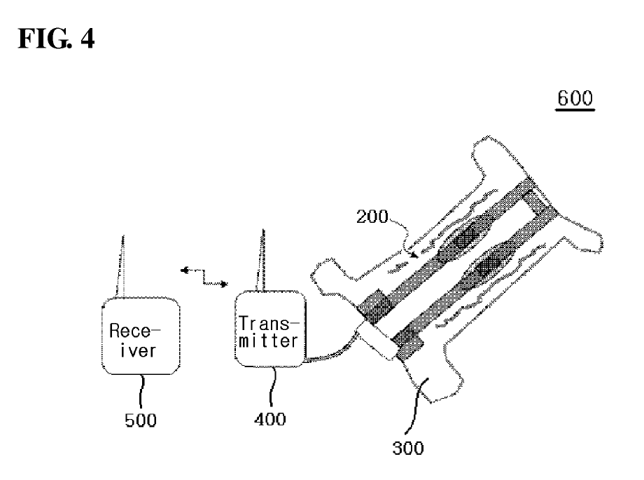

FIG. 4 is a schematic view showing an excreta detecting apparatus according to a third embodiment of the present invention. As shown in FIG. 4, an excreta detecting apparatus 600 includes the excreta detecting sensor 200, the diaper 300, a transmitter 400, and a receiver 500.

Although description has been made in that the excreta detecting sensor shown in FIG. 2a is applied for the illustrative purpose, the present invention is not limited thereto. In other words, the excreta detecting sensor shown in FIG. 1 may be applied.

When the excreta detecting sensor 200 is attached to the diaper 300, the attachment position is not limited to a specific region of the diaper 300. However, preferably, the excreta detecting sensor 200 is attached to the central portion of the diaper 200.

The excreta detecting sensor 200 may further have functions of sensing excreta based on a temperature and a contact resistance. In addition, the excreta detecting sensor 200 may further include sensors to measure bio-signals such as pulse and body temperature signals of the cared person.

The transmitter 400 receives the excreta detection signal from the excreta detecting sensor 200, creates excreta detection information for visible and acoustic notification of the fact that the excrement or the urine is detected, and transmits the excreta detection information to the receiver 500. In this case, the transmitter 400 transmits an identification number or an identification sign together with the excreta detection information so that the excreta detection information can be distinguished from a signal or information of other transmitters.

The receiver 500 wirelessly receives the excreta detection information received from the transmitter 400 to visibly or acoustically notify a carer of the fact that the cared person does defecation or urination.

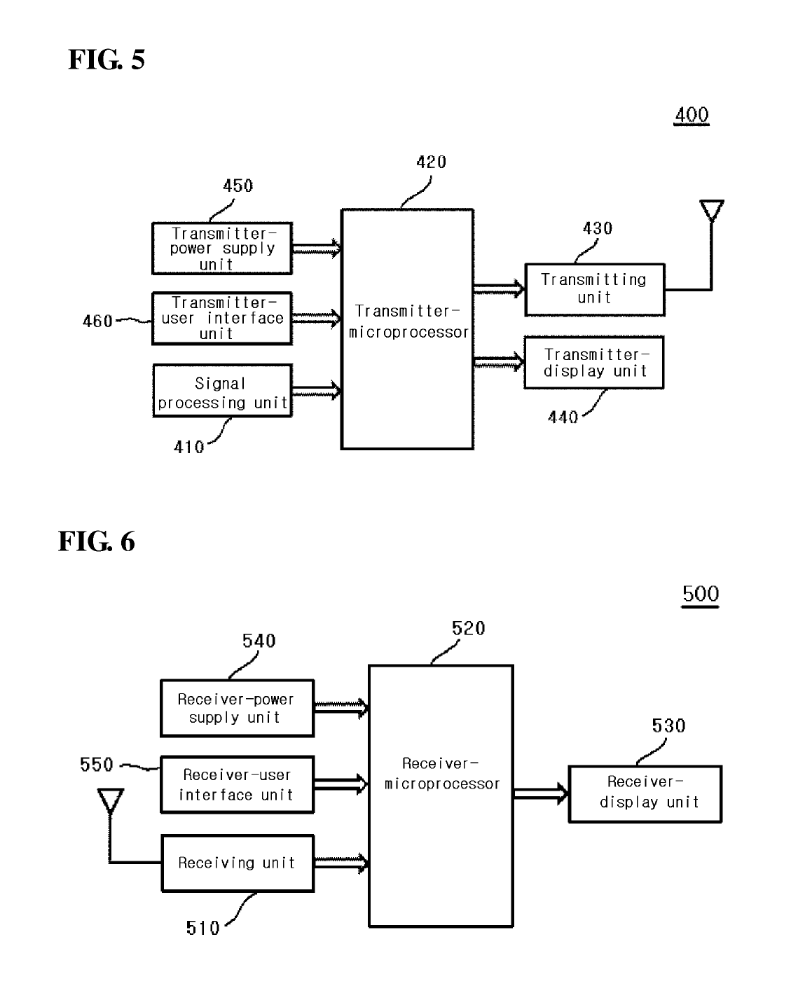

FIG. 5 is a block diagram showing the transmitter 400 according to one embodiment. The transmitter 400 includes a signal processing unit 410, a transmitter-microprocessor 420, a transmitting unit 430, a transmitter-display unit 440, a transmitter-power supply unit 450, and a transmitter-user interface unit 460.

The signal processing unit 410 receives the excreta detection signal output from the output terminal provided at the opposite side of the sensor connecting part 240 of the excreta detecting sensor 200 to amplify the excreta detection signal to a desirable level, or to attenuate a noise signal mixed with the excreta detection signal.

The transmitter-microprocessor 420 receives the excreta detection signal from the signal processing unit 410, detects a defecation number, a urination number, a defection period of time, and a urination period of time, and outputs the excreta detection information. In addition, the transmitter-microprocessor 420 controls the operations of parts provided in the transmitter 400.

The transmitting unit 430 wirelessly transmits the excreta detection information output from the transmitter-microprocessor 420.

The transmitter-display unit 440 displays the excreta detection information, which is output from the transmitter-microprocessor 420, on a display apparatus such a liquid crystal display (LCD) so that the carer can check the excreta detection near the diaper 300.

The transmitter-power supply unit 450 supplies power required for each part provided in the transmitter 400.

The transmitter-user interface unit 460 transmits information, which is input by a user through a user interface apparatus such as a touch panel, to the transmitter-microprocessor 420 in order to control the driving of the transmitter 400.

FIG. 6 is a block diagram showing the receiver 500 according to one embodiment. As shown in FIG. 6, the receiver 500 includes a receiving unit 510, a receiver-microprocessor 520, a receiver-display unit 530, a receiver-power supply unit 540, and a receiver-user interface unit 550.

The receiving unit 510 receives the excreta detection information wirelessly transmitted from the transmitting unit 430 of the transmitter 400 to amply the excreta detection information to a desirable level or to attenuate a noise signal mixed with the excreta detection information.

The receiver-microprocessor 520 receives the excreta detection signal from the receiving unit 510 to display the defecation number, the urination number, the defection period of time, and the urination period of time on the receiver-display unit 530. In addition, the receiver-microprocessor 520 controls the operation of each part provided in the receiver 500.

The receiver-power supply unit 540 supplies power required for each part provided in the receiver 500.

The receiver-user interface unit 550 transmits information, which is input by the user through a user interface apparatus such as a touch panel, to the receiver -microprocessor 520 in order to control the driving of the receiver 500.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

1. An excreta detecting sensor employing a fiber conductive line, comprising:
a plurality of fiber conductive lines arranged in parallel to each other while being spaced apart from each other by a predetermined interval;
a plurality of superior absorbent polymer sheets spaced apart from each other by a predetermined distance, and arranged on or under the fiber conductive lines perpendicularly to the fiber conductive lines while making contact with the fiber conductive lines, so that each fiber conductive line is changed to a conductive state or maintained in an insulating state depending on defecation or urination of a diaper wearer;
a power terminal connected with one of the fiber conductive lines to output excreta detection signal resulting from the defecation or the urination; and
an output terminal connected with at least one of remaining fiber conductive lines.

2. The excreta detecting sensor of claim 1, wherein an insulation degree between the fiber conductive lines is determined depending on an amount of moisture contained in the superior absorbent polymer sheets depending on the defecation or urination of the diaper wearer.

3. The excreta detecting sensor of claim 1, wherein superior absorbent polymer having predetermined absorbent or more is applied to surfaces of the superior absorbent polymer sheets.

4. The excreta detecting sensor of claim 1, wherein silver chloride (AgCl) is coated on contact surfaces between the superior absorbent polymer sheets and the fiber conductive lines before the superior absorbent polymer sheets are arranged.

5. An excreta detecting sensor employing a fiber conductive line, comprising:
a first fiber conductive line part including superior absorbent polymer which connects first and second fiber conductive lines having predetermined elasticity or more with each other and is gelated by moisture resulting from defecation or urination of a diaper wearer to disconnect the first and second fiber conductive lines from each other;
a second fiber conductive line part having a same structure as a structure of the first fiber conductive line part;
a fiber conductive line connecting part to connect the second fiber conductive line of the first fiber conductive line part with a second fiber conductive line of the second fiber conductive line part; and
a sensor connecting part comprising a power supply unit connected with the first fiber conductive line of the first fiber conductive line part and an excreta detection signal output terminal connected with the first fiber conductive line of the second fiber conductive line part.

6. The excreta detecting sensor of claim 5, wherein the first and second fiber conductive lines are fabricated through a process identical to a scheme of fabricating cloth having a spandex characteristic.

7. The excreta detecting sensor of claim 5, wherein the superior absorbent polymer is attached to a central portion of an outer skin of a diaper.

8. The excreta detecting sensor of claim 5, wherein the first and second fiber conductive line parts are attached to an outer skin of diaper in a state that the first and second fiber conductive line parts are elongated by a predetermined length or more.

9. An excreta detecting apparatus employing a fiber conductive line, comprising:
an excreta detecting sensor that detects moisture resulting from defecation or urination of a diaper wearer to output an excreta detection signal according to moisture detection;
a transmitter that creates excreta detection information from the excreta detection signal output from the excreta detecting sensor to wirelessly transmit the excreta detection information; and
a receiver that receives the excreta detection information from the transmitter and visibly or acoustically represents the defecation or urination of the diaper wearer.

10. The excreta detecting apparatus of claim 9, wherein the excreta detecting sensor comprises:
a plurality of fiber conductive lines arranged in parallel to each other while being spaced apart from each other by a predetermined interval;
a plurality of superior absorbent polymer sheets spaced apart from each other by a predetermined distance, and arranged on or under the fiber conductive lines perpendicularly to the fiber conductive lines while making contact with the fiber conductive lines, so that each fiber conductive line is changed to a conductive state or maintained in an insulating state depending on defecation or urination of a diaper wearer;
a power terminal connected with one of the fiber conductive lines to output excreta detection signal resulting from the defecation or the urination; and
an output terminal connected with at least one of remaining fiber conductive lines.

11. The excreta detecting apparatus of claim 9, wherein the excreta detecting sensor comprises:
a first fiber conductive line part including superior absorbent polymer which connects first and second fiber conductive lines having predetermined elasticity or more with each other and is gelated by moisture resulting from defecation or urination of a diaper wearer to disconnect the first and second fiber conductive lines from each other;
a second fiber conductive line part having a same structure as a structure of the first fiber conductive line part;
a fiber conductive line connecting part to connect the second fiber conductive line of the first fiber conductive line part with a second fiber conductive line of the second fiber conductive line part; and
a sensor connecting part comprising a power supply unit connected with the first fiber conductive line of the first fiber conductive line part and an excreta detection signal output terminal connected with the first fiber conductive line of the second fiber conductive line part.

12. The excreta detecting apparatus of claim 9, wherein the transmitter comprises:
a signal processing unit that receives the excreta detection signal output from the excreta detecting sensor to amplify the excreta detection signal to a desirable level or to attenuate a noise signal mixed with the excreta detection signal;
a transmitter-microprocessor that receives the excreta detection signal from the signal processing unit, detects information of a defecation number, a urination number, a defection period of time, and a urination period of time, and outputs excreta detection information according to the information;
a transmitting unit that wirelessly transmits the excreta detection information output from the transmitter-microprocessor; and
a transmitter-display unit that displays the excreta detection information output from the transmitter-microprocessor.

13. The excreta detecting apparatus of claim 9, wherein the receiver comprises:
a receiving unit that receives the excreta detection information transmitted from the transmitter to amply the excreta detection information to a desirable level or to attenuate a noise signal mixed with the excreta detection information; and
a receiver-microprocessor that receives the excreta detection information from the receiving unit and displays information of a defecation number, a urination number, a defection period of time, and a urination period of time on a receiver-display unit.
